(19) United States
(12) Patent Application Publication
(10) Pub. No.: US 2024/0382325 A1
(43) Pub. Date: Nov. 21, 2024

(54) TEMPORARY INTRAVASCULAR SCAFFOLDS FOR THE TREATMENT OF RESIDUAL STENOSIS FOLLOWING BALLOON ANGIOPLASTY

(71) Applicant: EFEMORAL MEDICAL, INC., Los Altos, CA (US)

(72) Inventors: LEWIS B. SCHWARTZ, Lake Forest, IL (US); IVAN TZVETANOV, Mountain View, CA (US); ALEX ESTRADA, Menlo Park, CA (US)

(21) Appl. No.: 18/692,289

(22) PCT Filed: Sep. 19, 2022
(86) PCT No.: PCT/US2022/043920

§ 371 Date: Mar. 14, 2024

Related U.S. Application Data
(60) Provisional application No. 63/245,652, filed on Sep. 17, 2021.

(51) Int. Cl. A61F 2/91; A61F 2/82; A61F 2/958

(52) U.S. Cl. CPC A61F 2/91; A61F 2/958; A61F 2002/825; A61F 2210/0004; A61F 2220/0025; A61F 2230/0069; A61F 2240/00; A61F 2250/0067

(57) ABSTRACT

Devices, systems, and methods are provided to maintain or enhance blood flow through the blood vessel. Balloon-expandable, bioresorbable, vascular stent elements provide high initial radial force at the vessel wall in order to treat residual stenosis and dissection following balloon angioplasty and then slowly soften and degrade over time following implantation.

### CROSS REFERENCES TO RELATED APPLICATION

This application claims the benefit and priority of U.S. Provisional Patent Application No. 63/245,652, entitled “TEMPORARY INTRAVASCULAR SCAFFOLDS FOR THE TREATMENT OF RESIDUAL STENOSIS FOLLOWING BALLOON ANGIOPLASTY”, filed on Sep. 17, 2021, the full disclosure of the above referenced application is incorporated herein by reference.

### FIELD OF THE INVENTION

The present application pertains generally to the field of medical devices. More specifically, the present application pertains to the design and manufacture of intravascular stents intended to maintain patency (blood flow) of blood vessels (arteries and veins).

### BACKGROUND

Percutaneous peripheral intervention (PPI) has become the treatment-of-choice for symptomatic peripheral arterial occlusive disease (PAOD). This minimally invasive therapy provides equivalent pain relief and limb salvage as compared to surgical bypass grafting while minimizing patient morbidity, complications and cost. Unfortunately, its durability remains poor; after only one-year, approximately 50% of all PPI procedures will be attended by symptomatic recurrence and/or restenosis necessitating reintervention. In one recent study of the percutaneous treatment of long femoropopliteal occlusive lesions (>150 mm) with balloon angioplasty, only 34% remained patent and free from restenosis after only one year.

In the current era, the mainstay of endovascular intervention for PAOD is percutaneous transluminal balloon angioplasty (PTA). Although generally successful in immediately restoring patency and improving arterial flow, balloon angioplasty is only marginally effective as (1) it rarely restores the target lesion to its full, original diameter, and (2) the majority of treated vessels mechanically recoil and re-narrow over the first few months. In order to more widely dilate the target lesion, smooth dissections, minimize residual stenosis and afford more sustained patency, percutaneously deliverable metal scaffolds or “stents” were developed in the 1990s. The first stents to become widely available were “balloon-expandable”: rigid, meshed tubes of stainless steel that, when crimped onto angioplasty balloons, could be advanced through the arterial tree coaxially and deployed via balloon inflation to abut arterial plaque. Unfortunately, the rigidity of metallic (stainless steel or cobalt chrome) balloon-expandable stents (BES) limits their applicability; only very short devices can be safely implanted into the leg as long devices would be crushed and bent when the patient walked or sat. Hence, while occlusive atherosclerotic lesions in lower extremities >30 cm are frequently encountered in clinical practice, the longest commercially available BES is only 6 cm in length.

To create stents that were more flexible, an equiatomic alloy of nickel-titanium (nitinol) used in the aerospace industry was adapted for human medical application in the 1990s. Nitinol exhibits the properties of superelasticity and shape memory such that the device can be manufactured in a miniaturized, compressed state then re-expanded to its original dimensions in the warm environs of the human vasculature. The result is a long, flexible, metal stent appropriate for implantation into vessels that bend and twist. Early studies of the device suggested that nitinol self-expanding stents (SES) exhibited superior patency compared with balloon dilation alone and, indeed, slotted-tube nitinol SES have become the most commonly used stents for this clinical application.

Unfortunately, nitinol SES have several critical pitfalls. First, their relatively poor radial strength leads to chronic under-expansion in hardened vessels. Indeed, in one study, post-procedure residual stenosis following SES implantation into calcified arteries was 70%. Second, the chronic outward force exerted by these ever-expanding, permanent devices continually stimulates inflammation, foreign body reaction, smooth muscle cell proliferation and restenosis. This phenomenon, known as “neointimal hyperplasia” or “vascular proliferative disorder”, is particularly prevalent in long arteries prone to bending and twisting. Finally, nitinol stents' flexibility is offset by their disquieting tendency towards weakness, fatigue and fracture. Reported to be as high as 65%, SES fracture and transection is clearly associated with restenosis and therapeutic failure.

Thus, the two most popular devices used to restore patency to human peripheral arteries, namely PTA and SES, are both woefully inadequate. Because they provide only temporary or weak radial support, they are unable to fully dilate the target lesion at the time of the procedure. The “residual stenosis” that is left untreated can be significant.

In general, both PTA and SES routinely “leave behind” 15-25% stenosis within the treated artery. Indeed, the definition of “technical success” in these studies is residual stenosis <30%. In contrast, the high-radial-strength design of metal BES assures that the offending lesion will be fully expanded. When measured, residual stenosis following implantation of BES in the femoropopliteal arterial system is only ˜3%.

Therefore, it would be advantageous to have a stent that can be safely used in highly mobile vasculature with less residual stenosis. At least some of these objectives will be met by the embodiments described below.

### SUMMARY

The embodiments herein describe a device for placement within a blood vessel to maintain or enhance blood flow through the blood vessel. The device may comprise one or more balloon-expandable, bioresorbable, vascular stent elements configured to be implanted in the blood vessel as a stent. The stent may be configured to provide high initial radial force at the blood vessel wall in order to treat residual stenosis and dissection following balloon angioplasty. The stent may be configured to soften and degrade over time following implantation. In an embodiment, bending of the blood vessel is accommodated by bending of spaces between the stent elements. In an embodiment, the stent element comprises a therapeutic drug. The therapeutic drug may prevent or attenuate inflammation, cell dysfunction, cell activation, cell proliferation, neointimal formation, thickening, late atherosclerotic change and/or thrombosis. The stent elements may have a stent pattern configured to forego flexibility in order to achieve higher radial strength. The stent pattern may be configured to forego resistance to foreshortening and to utilize foreshortening within each stent element to achieve high radial force.

This and other aspects of the present disclosure are described herein.

### BRIEF DESCRIPTION OF THE DRAWINGS

Present embodiments have other advantages and features which will be more readily apparent from the following detailed description and the appended claims, when taken in conjunction with the accompanying drawings, in which:

FIG. 1 shows the typical radial resistive forces of intravascular stents.

FIG. 2A illustrates one embodiment of a multi-element stent. FIG. 2B is a magnified view of the stent elements in FIG. 2A.

FIGS. 3A-3C depict deployment of a balloon-expandable multi-element stent.

FIG. 4A shows an implanted multi-element stent in a popliteal artery during full flexion of the hip and knee. FIG. 4B depicts the implanted device of FIG. 4A shown in three dimensions.

FIG. 5 shows an embodiment of a stent pattern.

FIG. 6 shows a laser cut stent.

FIG. 7 shows radial resistive forces of commercialized self-expanding stents and balloon-expandable metal stents compared to a stent described herein.

FIG. 8 shows the angiographic appearance of an occlusive lesion within the left superficial femoral artery that was treated with balloon angioplasty.

FIG. 9 shows an embodiment of a stent pattern.

FIG. 10 is a schematic diagram of a micro-stereolithograph used to create a stent, according to one embodiment.

FIG. 11 shows post-procedural residual stenosis.

### DETAILED DESCRIPTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The word “exemplary” is used herein to mean “serving as an example, instance, or illustration.” Any implementation described herein as “exemplary” is not necessarily to be construed as advantageous over other implementations.

Various embodiments are described herein with reference to the figures. The figures are not drawn to scale and are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

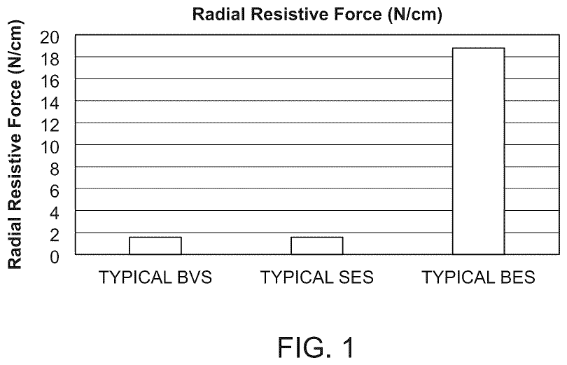

FIG. 1 shows the typical radial resistive forces of intravascular stents. A typical “bioresorbable vascular scaffold” (BVS) or absorbable stent has a radial resistive force of under 2 N/cm. Similarly, a typical self-expanding metal stent (SES) has a radial resistive force of under 2 N/cm. Typical balloon-expandable metal stents (BES) have a much higher radial resistive force, sometimes above 18 N/cm.

The embodiments herein describe the design of a balloon-expandable, intravascular stent system that temporarily provides high radial force at the arterial wall (in order to minimize residual stenosis) then slowly softens and degrades following arterial healing. A critical design element of the individual stent elements is the provision of radial strength more typical of highly effective, rigid, balloon-expandable metal stents as opposed to weaker self-expanding metal stents.

In contrast to most stent patterns which are designed to marry radial force, flexibility, and resistance to foreshortening, the patterns described herein are specifically tailored to maximize radial force and forego flexibility. In some embodiments they may also resist foreshortening within an individual stent element in order to further maximize radial force. In a device made up of multiple, serial stent elements, significant foreshortening within each stent element does not lead to significant foreshortening overall.

The devices described herein are multi-element, vascular stents (or “vascular scaffolds”). These stents are comprised of multiple, short, rigid, cylindrical stent segments, or elements, which are separate from one another but may be referred to together as a multi-element stent.

Generally, at least two of the elements of the multi-element stent described herein will be sufficiently rigid to provide a desired level of strength to withstand the stresses of the vessel in which they are placed, such as a tortuous peripheral vessel. At the same time, a multi element stent will also be flexible, due to the fact that it is made up of multiple separate elements, thus allowing for placement within a curved, torturous blood vessel. In some embodiments, at least two of the elements vary in rigidity or radial strength in a multi-element stent. In one embodiment, the outer elements may have a lesser radial strength than the inner elements in a multi-element stent. In another embodiment, a multi-element stent comprises elements having an increasing radial strength serially along the length of the multi-element stent, such as in an AV fistula. Thus, the radial strength of elements may vary and be tailored by known characteristics of a target artery.

Additionally, the multi element stents described herein will usually be balloon-expandable rather than self-expanding, since balloon-expandable stents are typically stronger than self-expanding stents. Each balloon expandable element of the stent may have relatively high radial force (rigidity) due to the described structures and materials. A stent element is defined as being radially rigid if it has a radial strength significantly higher than self-expanding stents that is similar or greater in magnitude to that of traditional, metal balloon-expandable stents, such as those made of steel or cobalt-chromium.

When mounted serially on an inflatable balloon, they can be simultaneously implanted side-by-side in long blood vessels. During motion of the organism, the elements can move independently, maintaining their individual shape and strength while the intervening, non-stented elements of the vessel can twist, bend and rotate unencumbered. The result is a treated vessel with a rigidly maintained flow channel that still enjoys unrestricted flexibility during organismal movement.

The described embodiments exploit the principles that, (1) a rigid device that is deployed via balloon-expansion represents the optimal design of an intravascular stent given its transient effect on the arterial wall and relative ease of precise implantation, (2) a long, rigid device cannot be safely implanted in an artery that bends and twists with skeletal motion, (3) long arteries that bend and twist could be effectively treated with multiple, short BES that allow the intervening, non-stented arterial elements to move unencumbered, (4) the length, number and spacing of the stent elements could be determined by the known and predictable bending characteristics of the target arteries, and (5) arteries need only be scaffolded transiently; late dissolution of the stent will have little effect on the long-term effectiveness of treatment.

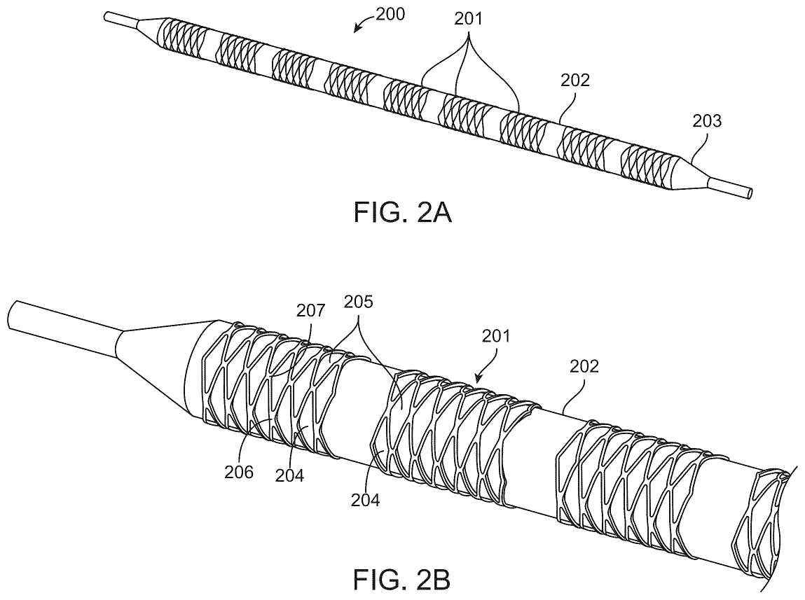

One embodiment of the fully assembled device in shown in FIG. 2A. A single balloon inflation and device deployment can treat a long segment of diseased artery while still preserving the critical ability of the artery to bend with skeletal motion such as sitting or walking. Multi-element stent 200 comprises multiple stent elements 201. Individual balloon-expandable stent elements 201 are crimped onto an inflatable balloon 203 to facilitate delivery. FIG. 2B is a magnified view of the stent elements 201 in FIG. 2A. Individual elements 201 are positioned serially along a longitudinal length of the balloon 203 and spaced such that the stent elements 201 do not touch one another. Further, the spacing is such that after deployment, the stent elements 201 do not touch or overlap during skeletal movement. The number of elements 201, length of elements, and gap 202 between elements 201 may vary depending on the target vessel location. In an embodiment, each element 201 in the multi-element stent 200 has the same length. In multi-element stents having three or more elements 201, and thus two or more gaps 202, the gaps may be of the same length.

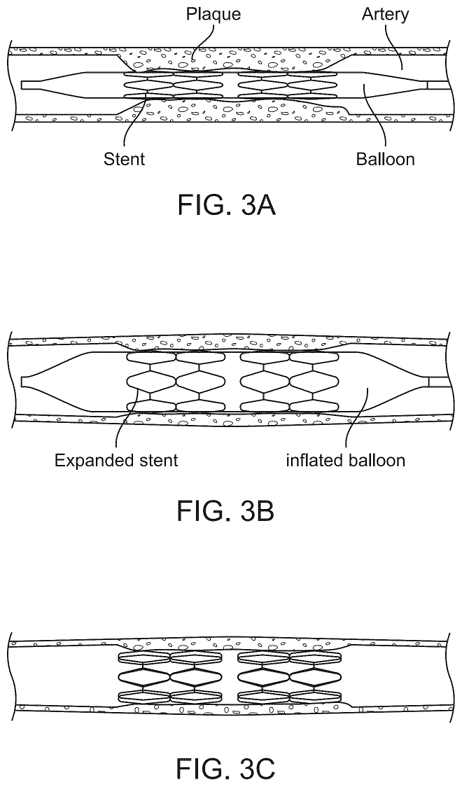

FIGS. 3A-3C depict deployment of a balloon-expandable multi-element stent. In FIG. 3A a multi-element stent mounted on a balloon is advanced to the lesion. In FIG. 3B the balloon and stent are expanded. In FIG. 3C the balloon is withdrawn leaving the multi-element stent still within the artery.

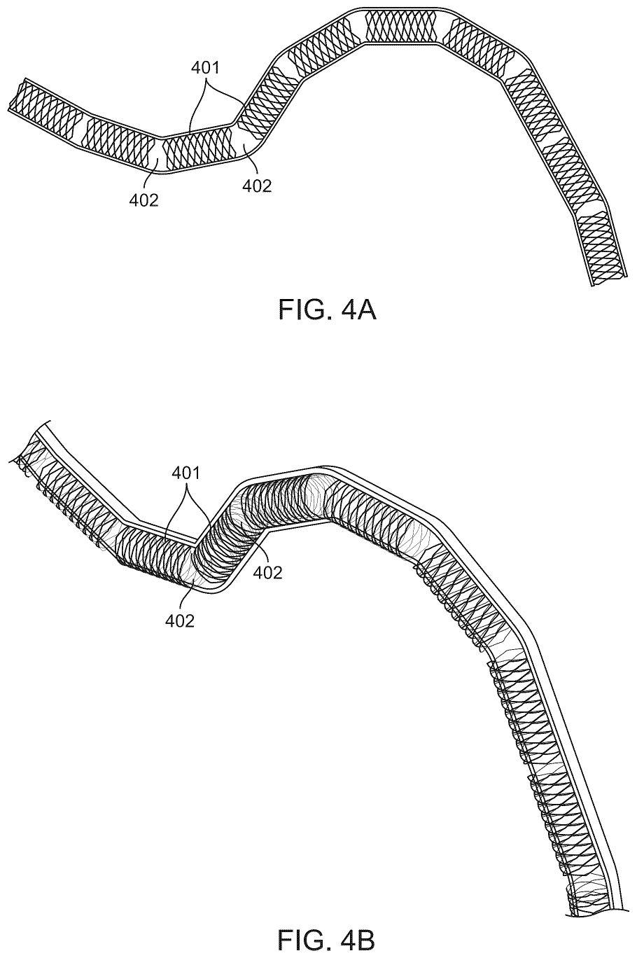

FIG. 4A shows an implanted multi-element stent in a popliteal artery during full flexion of the hip and knee. FIG. 4B depicts the implanted device of FIG. 4A shown in three dimensions. The individual stent elements 401 are spaced such that they do not overlap even when the artery is highly bent. Unencumbered arterial movement is afforded through flexion or extension of the unstented gaps 402.

Stent elements may comprise various shapes and configurations. Some or all of the stent elements may comprise closed-cell structures formed by intersecting struts. Closed-cell structures may comprise diamond, rhombus, rhomboid, trapezium, kite, square, rectangular, parallelogrammatic, triangular, pentagonal, hexagonal, heptagonal, octagonal, clover, lobular, circular, elliptical, and/or ovoid geometries. Closed-cells may also comprise slotted shapes such as H-shaped slots, I-shaped slots, J-shaped slots, and the like. Additionally or alternatively, stent may comprise open cell structures such as spiral structures, serpentine structures, zigzags structures, etc. Strut intersections may form pointed, perpendicular, rounded, bullnosed, flat, beveled, and/or chamfered cell corners. In an embodiment, stent may comprise multiple different cells having different cell shapes, orientations, and/or sizes. Various cell structures have been described in PCT International Application Number PCT/US16/20743, entitled “MULTI-ELEMENT BIORESORBABLE INTRAVASCULAR STENT”, PCT International Application Number PCT/US20/19132, entitled “ABSORBABLE INTRAVASCULAR DEVICES THAT EXHIBIT THEIR GREATEST RADIAL STRENGTH AT THEIR NOMINAL DIAMETERS”, and PCT International Application Number PCT/US19/35861, entitled “ABSORBABLE INTRAVASCULAR DEVICES THAT SHORTEN UPON EXPANSION CREATING SPACE FOR VASCULAR MOVEMENT”, the full disclosures of which are herein incorporated by reference.

Returning to FIG. 2B, in this exemplary embodiment, the stent elements 201 have a diamond or rhombus shaped closed-cell pattern. Elements 201 comprise intermixed diamond shaped closed cells 204, 205. The stent elements may have cell patterns with relatively thick strut widths and obliquely-angled links. Elements 201 may comprise wide struts 206 of 225 microns or larger. Elements 201 may similarly comprise thick struts 206 of 225 microns or larger. In an embodiment, elements 201 comprise struts 206 with a width and/or thickness of approximately 250 microns. Diamond shaped cells 204 may be aligned in the longitudinal and/or the circumferential directions in a repeating pattern. Similarly, diamond shaped cells 205 may be aligned in the longitudinal and/or the circumferential directions in a repeating pattern. Additionally or alternatively, diamond shaped cells 204 and diamond shaped cells 205 may be helically aligned in an alternating pattern. In an embodiment, diamond shaped cells 204 and diamond shaped cells 205 are circumferentially offset. Additionally, diamond shaped cells 205 may be formed at a central location between four adjacent diamond shaped cells 204. The width of struts 206 between two corners of longitudinally aligned diamond shaped cells 204 are larger than the width of struts 207 between two corners of longitudinally aligned diamond shaped cells 205.

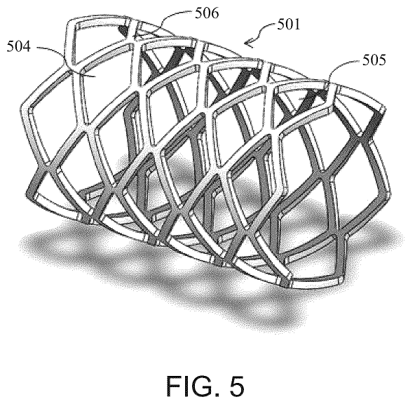

One embodiment of a stent pattern is shown in a single stent element 501 in FIG. 5. Its strength is imparted through a design composed of tightly closed cells 504, 505 with relatively thick struts 506. When compressed radially (crimped) onto a balloon, the struts 506 are oriented axially (along the length of the blood vessel). When expanded, however, the struts 506 become oriented with the vessel's diameter, and, like the columns of a building, lend additional resistance to the circumferential compression forces acting to collapse the blood vessel. The closed cell configuration also spreads the compressive load throughout the repeating structure making it highly resistant to deformation. This particular embodiment of a closed cell configuration pattern is attended by significant foreshortening during expansion. This foreshortening further concentrates the struts 506 into a smaller area and increases strength.

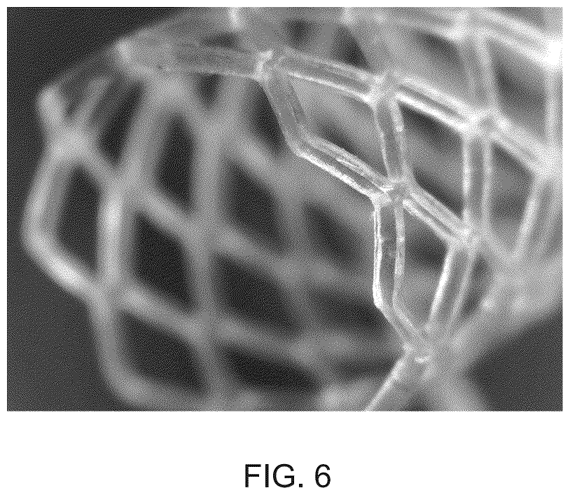

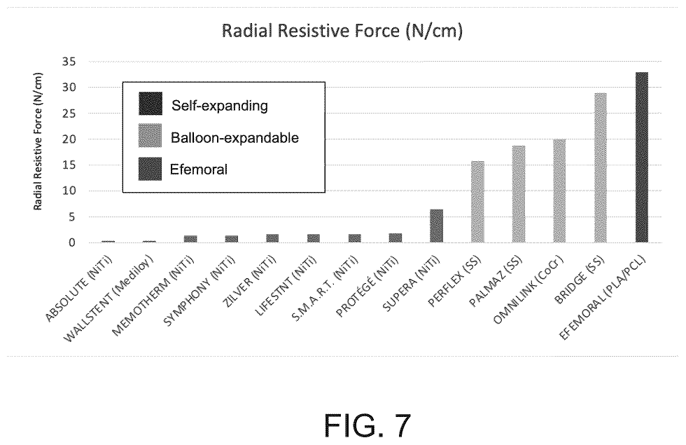

An example of a manufactured, laser-cut polymer scaffold of is shown in in FIG. 6. Its novel polymer and closed-cell pattern supports a radial resistive force that is equal or better than comparable nitinol (NiTi), 316L stainless steel (SS) or cobalt-chromium (CoCr) devices as is shown in FIG. 7. Radial resistive force of commercialized self-expanding (bars 1-9 on the left) and balloon-expandable (bars 10-13 in the middle) metal stents are shown compared to the Efemoral resorbable scaffold (bar 14 on the right) described herein. Note that, although the Efemoral scaffold is comprised of a bioresorbable polymer, it is as strong or stronger than popular peripheral metal stents. This is achieved while still maintaining a strut thickness which is only slightly greater than the thickness of popular peripheral metal stents such as EverFlex (228 μm, Medtronic; Minneapolis, MN), Innova (213 μm, Boston Scientific; Marlborough, MA), Omnilink (210 μm, Abbott Laboratories; Abbott Park, IL) and S.M.A.R.T. (200 μm, Cardinal Health, Dublin, OH).

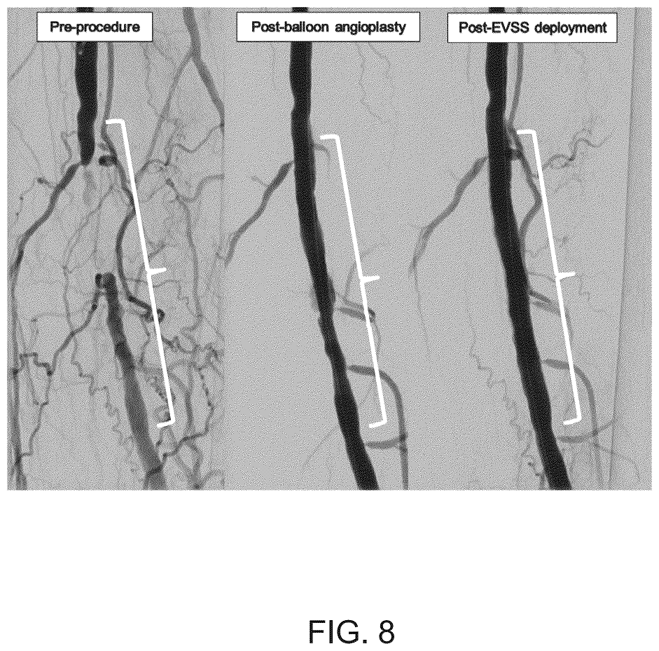

That this resorbable, balloon-expandable device would enlarge the arterial lumen (and relieve residual stenosis) during balloon angioplasty was demonstrated in humans. FIG. 8 shows the angiographic appearance of an occlusive lesion within the left superficial femoral artery that was treated with balloon angioplasty. Although the procedure restored patency to the artery, the result was suboptimal including leaving residual stenosis >50% and traumatic dissection. Both of these complications were effectively treated by immediate implantation of the resorbable scaffold described herein. Angiographic images of a 75 year-old male claudicant. The patient's occlusive lesion in the left superficial femoral artery is shown pre-procedure (left panel), following balloon dilatation (center panel) and following EVSS deployment (right panel). The white bracket indicates the target lesion. Note the residual stenosis and dissection following balloon angioplasty (center panel) that has been effectively treated by EVSS deployment (right panel).

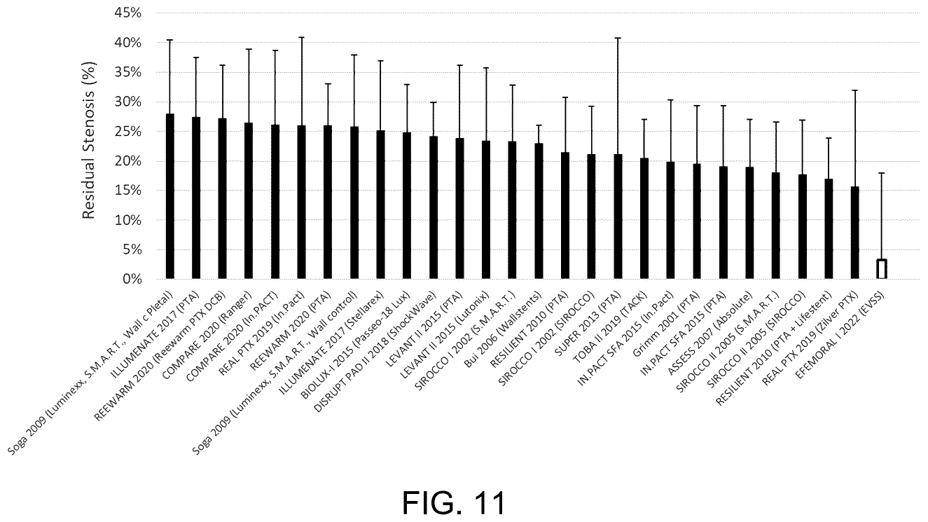

The effectiveness of the stents describe herein for the treatment of residual stenosis at the time of percutaneous vascular intervention is illustrated by results of the EFEMORAL I clinical trial. The purpose of the EFEMORAL I Clinical Investigation is to evaluate the safety and performance of the sirolimus-eluting Efemoral Vascular Scaffold System (EVSS) in patients with symptomatic peripheral arterial occlusive disease from stenosis or occlusion of the femoropopliteal artery. To date, ten subjects have been enrolled in EFEMORAL I. Their mean age was 75±8 years; 80% were male and all presented with lifestyle-limiting claudication (Rutherford-Becker Category 2 or 3) with a mean ankle-brachial index of 0.74±0.15. All had stenosis (n=6) or occlusion (n=4) within the mid- or distal superficial femoral artery (SFA, n=9) or external iliac artery (n=1) with a mean diameter stenosis of 90%±15% in target lesions measuring 5.4±2.0 cm. After wire crossing, all were treated with standard balloon angioplasty immediately followed by implantation of a 6 mm×60 mm, 5-scaffold, EVSS loaded with sirolimus. As expected, implantation of the balloon-expandable EVSS significantly enhanced target artery lumen size, reduced residual stenosis and smoothed barotrauma-induced dissection following balloon angioplasty. In these 10 patients, the mean residual stenosis of 44±12% following balloon angioplasty was reduced to 3.2±15% following EVSS implantation. The mean post-procedure residual stenosis of 3.2% is the lowest ever reported in clinical trials of femoropopliteal intervention (FIG. 11). FIG. 11 shows post-procedural residual stenosis in the EFEMORAL I clinical trial (open bar) compared with historical trials of percutaneous femoropopliteal vascular intervention (closed bars). The result is that the treated artery is rendered luminally larger at the time of the procedure and, therefore, less prone to restenosis and/or thrombosis over time.

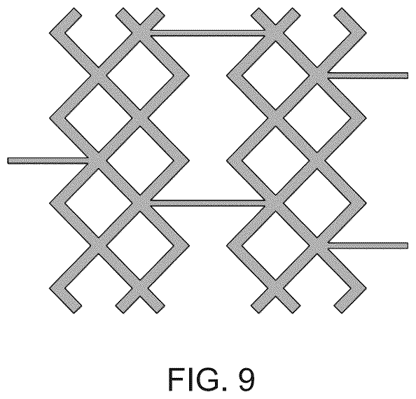

Another embodiment, depicted as an unfolded single stent element in FIG. 9, is also tailored to maximize radial force while foregoing flexibility. However, it contains connectors that limit foreshortening with expansion which may be useful in certain arteries and lesions. In this manner, the degree of foreshortening can be tailored to specific anatomic needs by combining elements of different patterns similar to the method shown.

The stents described herein may be formed from various different materials. In an embodiment, stents may be formed a polymer or co-polymer. In various alternative embodiments, the stent or stent element may be made from any suitable bioresorbable material such that it will dissolve non-toxically in the human body, such as but not limited to polyesters such as Polylactic acid, Poly(ε-caprolactone), Polyglycolic acid, and Polyhydroxyalkanoate, amino acid based polymers such as Polyesteramide, polycarbonates such as Polytrimethylene carbonate as well as any and all copolymers of the types described herein. In alternative embodiments, the stents may be formed from a permanent material such as a metal.

In various embodiments, any suitable polymer or copolymer may be used to construct the stent. The term “polymer” is intended to include a product of a polymerization reaction inclusive of homopolymers, copolymers, terpolymers, etc., whether natural or synthetic, including random, alternating, block, graft, branched, cross-linked, blends, compositions of blends and variations thereof. The polymer may be in true solution, saturated, or suspended as particles or supersaturated in the beneficial agent. The polymer can be biocompatible, or biodegradable. For purpose of illustration and not limitation, the polymeric material may include, but is not limited to, L-lactide, poly(D-lactic acid) (PDLA), poly(D,L-lactic acid) (PDLLA), poly(iodinated desamino tyrosyl-tyrosine ethyl ester) carbonate, poly(lactic-co-glycolic acid) (PLGA), poly(iodinated desaminotyrosyl-tyrosine ethyl ester) carbonate, salicylate based polymer, semicrystalline polylactide, phosphorylcholine, 8-caprolactone, polycaprolactone (PCL), poly-D,L-lactic acid, poly-L-lactic acid, poly(lactideco-glycolide), poly(hydroxybutyrate), poly(hydroxybutyrate-co-valerate), polydioxanone (PDS), polyorthoester, polyanhydride, poly(glycolic acid), poly(glycolic acid-co-trimethylene carbonate), polyphosphoester, polyphosphoester urethane, poly(amino acids), cyanoacrylates, poly(trimethylene carbonate), poly(iminocarbonate), polyalkylene oxalates, polyphosphazenes, polyiminocarbonates, and aliphatic polycarbonates, fibrin, fibrinogen, cellulose, starch, collagen, polyurethane including polycarbonate urethanes, polyethylene, polyethylene terephthalate, ethylene vinyl acetate, ethylene vinyl alcohol, silicone including polysiloxanes and substituted polysiloxanes, polyethylene oxide, polybutylene terephthalate-co-PEG, PCL-co-PEG, PLA-co-PEG, PLLA-co-PCL, polyacrylates, polyvinyl pyrrolidone, polyacrylamide, and combinations thereof. Non-limiting examples of other suitable polymers include thermoplastic elastomers in general, polyolefin elastomers, EPDM rubbers and polyamide elastomers, and biostable plastic material including acrylic polymers, and its derivatives, nylon, polyesters and expoxies. In some embodiments, the stent may include one or more coatings, with materials like poly-L-lactide (PLLA) or poly(D,L-lactic acid) (PDLLA). These materials are merely examples, however, and should not be seen as limiting the scope of the invention. The coating may comprise a drug and a solvent capable of dissolving the drug and swelling or softening the scaffold structural polymer. The solvent may be any single solvent or a combination of solvents. For purpose of illustration and not limitation, examples of suitable solvents include water, aliphatic hydrocarbons, aromatic hydrocarbons, alcohols, ketones, dimethyl sulfoxide, tetrahydrofuran, dihydrofuran, dimethylacetamide, acetonitrile, acetates, and combinations thereof.

The devices described herein may include a therapeutic drug or agent intended to prevent or attenuate pathologic consequences of intraluminal intervention such as inflammation, cell dysfunction, cell activation, cell proliferation, neointimal formation, thickening, late atherosclerotic change and/or thrombosis. Any suitable therapeutic agent (or “drug”) may be incorporated into, coated on, or otherwise attached to the stent, in various embodiments. In an embodiment, the drug may be Sirolimus and/or its derivatives. Examples of such therapeutic agents include, but are not limited to, antithrombotics, anticoagulants, antiplatelet agents, anti-lipid agents, thrombolytics, antiproliferatives, anti-inflammatories, agents that inhibit hyperplasia, smooth muscle cell inhibitors, antibiotics, growth factor inhibitors, cell adhesion inhibitors, cell adhesion promoters, antimitotics, antifibrins, antioxidants, anti-neoplastics, agents that promote endothelial cell recovery, matrix metalloproteinase inhibitors, anti-metabolites, antiallergic substances, viral vectors, nucleic acids, monoclonal antibodies, inhibitors of tyrosine kinase, antisense compounds, oligonucleotides, cell permeation enhancers, hypoglycemic agents, hypolipidemic agents, proteins, nucleic acids, agents useful for erythropoiesis stimulation, angiogenesis agents, anti-ulcer/anti-reflux agents, and anti-nauseants/anti-emetics, PPAR alpha agonists such as fenofibrate, PPAR-gamma agonists selected such as rosiglitazaone and pioglitazone, sodium heparin, LMW heparins, heparoids, hirudin, argatroban, forskolin, vapriprost, prostacyclin and prostacylin analogues, dextran, D-phe-pro-arg-chloromethylketone (synthetic anti-thrombin), glycoprotein IIb/IIIa (platelet membrane receptor antagonist antibody), recombinant hirudin, thrombin inhibitors, indomethacin, phenyl salicylate, beta-estradiol, vinblastine, ABT-627 (astrasentan), testosterone, progesterone, paclitaxel, methotrexate, fotemusine, RPR-101511A, cyclosporine A, vincristine, carvediol, vindesine, dipyridamole, methotrexate, folic acid, thrombospondin mimetics, estradiol, dexamethasone, metrizamide, iopamidol, iohexol, iopromide, iobitridol, iomeprol, iopentol, ioversol, ioxilan, iodixanol, and iotrolan, antisense compounds, inhibitors of smooth muscle cell proliferation, lipid-lowering agents, radiopaque agents, antineoplastics, HMG CoA reductase inhibitors such as lovastatin, atorvastatin, simvastatin, pravastatin, cerivastatin and fluvastatin, and combinations thereof.

Examples of antithrombotics, anticoagulants, antiplatelet agents, and thrombolytics include, but are not limited to, sodium heparin, unfractionated heparin, low molecular weight heparins, such as dalteparin, enoxaparin, nadroparin, reviparin, ardoparin and certaparin, heparinoids, hirudin, argatroban, forskolin, vapriprost, prostacyclin and prostacylin analogues, dextran, D-phe-pro-arg-chloromethylketone (synthetic antithrombin), dipyridamole, glycoprotein IIb/IIIa (platelet membrane receptor antagonist antibody), recombinant hirudin, and thrombin inhibitors such as bivalirudin, thrombin inhibitors, and thrombolytic agents, such as urokinase, recombinant urokinase, pro-urokinase, tissue plasminogen activator, ateplase and tenecteplase.

Examples of cytostatic or antiproliferative agents include, but are not limited to, rapamycin and its analogs, including everolimus, zotarolimus, tacrolimus, novolimus, ridafrolimus, temsirolimus, and pimecrolimus, angiopeptin, angiotensin converting enzyme inhibitors, such as captopril, cilazapril or lisinopril, calcium channel blockers, such as nifedipine, amlodipine, cilnidipine, lercanidipine, benidipine, trifluperazine, diltiazem and verapamil, fibroblast growth factor antagonists, fish oil (omega 3-fatty acid), histamine antagonists, lovastatin, topoisomerase inhibitors, such as etoposide and topotecan, as well as antiestrogens such as tamoxifen.

Examples of anti-inflammatory agents include, but are not limited to, colchicine and glucocorticoids, such as betamethasone, cortisone, dexamethasone, budesonide, prednisolone, methylprednisolone and hydrocortisone. Non-steroidal anti-inflammatory agents include, but are not limited to, flurbiprofen, ibuprofen, ketoprofen, fenoprofen, naproxen, diclofenac, diflunisal, acetominophen, indomethacin, sulindac, etodolac, diclofenac, ketorolac, meclofenamic acid, piroxicam and phenylbutazone.

Examples of antineoplastic agents include, but are not limited to, alkylating agents including altretamine, bendamucine, carboplatin, carmustine, cisplatin, cyclophosphamide, fotemustine, ifosfamide, lomustine, nimustine, prednimustine, and treosulfin, antimitotics, including vincristine, vinblastine, paclitaxel, docetaxel, antimetabolites including methotrexate, mercaptopurine, pentostatin, trimetrexate, gemcitabine, azathioprine, and fluorouracil, antibiotics, such as doxorubicin hydrochloride and mitomycin, and agents that promote endothelial cell recovery such as estradiol.

Antiallergic agents include, but are not limited to, permirolast potassium nitroprusside, phosphodiesterase inhibitors, prostaglandin inhibitors, suramin, serotonin blockers, steroids, thioprotease inhibitors, triazolopyrimidine, and nitric oxide.

Stents may be manufactured using an additive or a subtractive method. In any of the described embodiments, stents or stent elements may be manufactured as a sheet and wrapped into cylindrical form. Alternatively, stents or stent elements may be manufactured in cylindrical form using an additive manufacturing process. In an embodiment, stents maybe formed by extruding a material into a cylindrical tubing. In some embodiments, a longer stent element, may be formed during the manufacturing process and then cut into smaller stent elements/elements to provide a multi-element stent. In an embodiment, stent tubing may be laser cut with a pattern to form a stent element.

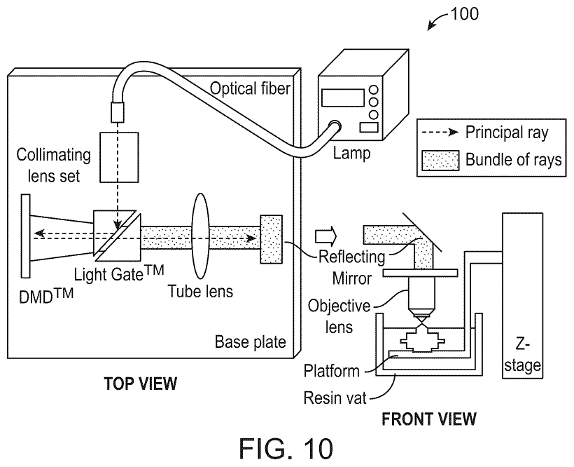

Referring now to FIG. 10, in one embodiment, stents may be manufactured using a micro-stereolithography system 100 (or “3D printing system”). Several examples of currently available systems that might be used in various embodiments include, but are not limited to: MakiBox A6, Makible Limited, Hong Kong; CubeX, 3D Systems, Inc., Circle Rock Hill, SC; and 3D-Bioplotter, (EnvisionTEC GmbH, Gladbeck, Germany).

The micro-stereolithography system may include an illuminator, a dynamic pattern generator, an image-former and a Z-stage. The illuminator may include a light source, a filter, an electric shutter, a collimating lens and a reflecting mirror that projects a uniformly intense light on a digital mirror device (DMD), which generates a dynamic mask. FIG. 10 shows some of these components of one embodiment of the micro-stereolithography system 100, including a DMD board, Z-stage, lamp, platform, resin vat and an objective lens. The details of 3D printing/micro-stereolithography systems and other additive manufacturing systems will not be described here, since they are well known in the art. However, according to various embodiments, any additive manufacturing system or process, whether currently known or hereafter developed, may potentially be used to fabricate stents within the scope of the present invention. In other words, the scope of the invention is not limited to any particular additive manufacturing system or process.

In one embodiment, the system 100 may be configured to fabricate stents using dynamic mask projection micro-stereolithography. In one embodiment, the fabrication method may include first producing 3D microstructural scaffolds by slicing a 3D model with a computer program and solidifying and stacking images layer by layer in the system. In one embodiment, the reflecting mirror of the system is used to project a uniformly intense light on the DMD, which generates a dynamic mask. The dynamic pattern generator creates an image of the sliced section of the fabrication model by producing a black-and-white region similar to the mask. Finally, to stack the images, a resolution Z-stage moves up and down to refresh the resin surface for the next curing. The Z-stage build subsystem, in one embodiment, has a resolution of about 100 nm and includes a platform for attaching a substrate, a vat for containing the polymer liquid solution, and a hot plate for controlling the temperature of the solution. The Z-stage makes a new solution surface with the desired layer thickness by moving downward deeply, moving upward to the predetermined position, and then waiting for a certain time for the solution to be evenly distributed.

Although particular embodiments have been shown and described, they are not intended to limit the invention. Various changes and modifications may be made to any of the embodiments, without departing from the spirit and scope of the invention. The invention is intended to cover alternatives, modifications, and equivalents.

What is claimed is:

1. A device for placement within a blood vessel to maintain or enhance blood flow through the blood vessel, the device comprising:
one or more balloon-expandable, bioresorbable, vascular stent elements configured to be implanted in the blood vessel as a stent;
wherein the stent is configured to provide high initial radial force at the blood vessel wall in order to treat residual stenosis and dissection following balloon angioplasty; and
wherein the stent is further configured to soften and degrade over time following implantation.

2. The device of claim 1, wherein bending of the blood vessel is accommodated by bending of spaces between the stent elements.

3. The device of claim 1, wherein the stent element comprises a therapeutic drug.

4. The device of claim 3, wherein the therapeutic drug prevents or attenuates inflammation, cell dysfunction, cell activation, cell proliferation, neointimal formation, thickening, late atherosclerotic change or thrombosis.

5. The device of claim 1, wherein the stent element has a stent pattern configured to forego flexibility in order to achieve higher radial strength.

6. The device of claim 5, wherein the stent pattern is configured to forego resistance to foreshortening and to utilize foreshortening within each stent element to achieve high radial force.
